(19) United States
(12) Patent Application Publication
(10) Pub. No.: US 2013/0103125 A1
(43) Pub. Date: Apr. 25, 2013

(54) DEVICE FOR TEMPERATURE REDUCTION

(71) Applicants: Andreas Radspieler, Neubeuern (DE); Klaus Fieback, Michendorf (DE); Dirk Carsten Büttner, Berlin (DE)

(72) Inventors: Andreas Radspieler, Neubeuern (DE); Klaus Fieback, Michendorf (DE); Dirk Carsten Büttner, Berlin (DE)

(73) Assignee: OTTO BOCK HEALTHCARE GMBH, Duderstadt (DE)

(21) Appl. No.: 13/696,391

(22) PCT Filed: May 10, 2011
(86) PCT No.: PCT/DE11/01050

§ 371 Date: Jan. 11, 2013

(30) Foreign Application Priority Data
May 11, 2010 (DE) 102010020262.2
Jan. 13, 2011 (DE) 202011001473.8

(51) Int. Cl. A61F 7/00

(52) U.S. Cl. CPC A61F 7/0085
USPC 607/104

(57) ABSTRACT

The invention discloses a device composed of PCM and of an actively cooling heat-exchanger system whose use in humans and animals reliably solves the previous problem of temperature build-up. The system can be configured in any strength, that is to say ranging from integration in flexible stockings to integration in orthosis stumps. This results in the extremely varied areas of application of the described technology, which range from PCMs in the form of modules in coolant channels or in the form of PCM-containing compounds, from which hoses are produced in which coolant can flow, to PCMs with heat exchangers that remove the built-up heat of the body.

The invention relates to a device for temperature reduction following the build-up of body heat.

It is known that a lot of sweat gathers at certain places in articles of use such as helmets, sneakers, car seats, bicycle saddles, riding saddles, and in textiles, i.e. flexible fabrics such as insoles or stockings, and not least in orthoses or prostheses for the body, the reason being that the heating of the body is intensive there and, comparatively speaking, can be removed only inadequately, in other words with body heat building up at the place in the article of use and/or in the flexible sheet material/the flexible matrix.

The problem is already tackled in a variety of ways. For example, in summer shoes that are intended to be closed, some of the upper material, for example leather, is interrupted by air holes, such that there is some movement of air in the shoe. The holes are in part covered by nets in order to give the impression of a closed shoe. The same thing is practiced in all types of helmets, where the helmet is simply interrupted at narrow points, or nets are introduced, so that cooling air can take up the heat given off by the body. A disadvantage of the air holes is that they interrupt the function of the helmet at the places in question and, furthermore, that at various places, for example on shoe soles or on prostheses, such interruption of the material would cause the entire part to be unusable. Air holes on textile upper parts are also offered by zippers under the armpits in the case of jackets, etc.

Moreover, there are storage textiles, for example shoe insoles, bed linen, etc., which however are without active removal of heat.

U.S. Pat. No. 5,722,482 discloses a textile sheet material with a phase change material (PCM). This sheet material is provided to buffer the body heat, that is to say it serves as a protection against cold in order to maintain the body's own heat over a longer period of time. A disadvantage of the flexible sheet material known from U.S. Pat. No. 5,722,482 is that no means are provided for removing the built-up body heat.

The object of the invention is to make available a device for temperature reduction that can be used in hard articles of use, for example prostheses, and also in bendable articles of use, for example soles of shoes, and in flexible articles of use, for example support stockings, which articles of use are in direct thermal contact with the body of a living being, wherein the device can be integrated into the article and/or the flexible matrix without the article and/or the flexible matrix losing its functionality.

The way in which the object is achieved is disclosed below in the description, the figures and the claims.

The general concept of the invention is that, with the aid of so-called PCMs (phase change materials with high heat storage capacity), for example in conjunction with corresponding heat exchangers on flexible and/or rigid articles that are in contact with the body, it is possible to form heat storage means that keep the surface temperature of the skin, or the contact temperature of the article lying on the skin, within a comfortable range and at the same time do not alter the wearing comfort and/or functionality of the article. The regeneration of the PCMs by a heat exchanger preferably takes place continuously, even during wearing.

Accordingly, the present invention relates to a device for temperature reduction, provided for articles lying on the skin and/or flexible matrices lying on the skin, which articles or flexible matrices are in direct thermal contact with the body of a living being, wherein the device is at least partially integrated in the article and/or the flexible matrix and comprises a phase change material (PCM) and a heat exchanger. The invention also relates to the use of PCMs in pure form and/or in the form of compounds, e.g. with plastic polymers, silicates, graphites, etc., coupled to a heat exchanger, in articles such as orthoses and/or prostheses, in items of clothing, in shoes, helmets, saddles or seats, and also in flexible matrices such as insoles and/or stockings.

The device according to the invention for temperature reduction is, for example, contained or embodied only in part of the article of use or of the orthopedic article or of the soft-tissue-stabilizing fabric (stocking). The article of use or the orthopedic article differs from the flexible fabric mainly in that it either has a non-flexible form or has at least a form that is solid in parts. As regards the terms used, the two “categories” cannot be separated from each other, since an article can partially have a flexible form and a fabric can have solid parts. It is therefore possible that the device according to the invention is integrated partially or entirely in a flexible area of an otherwise non-flexible article, and conversely that the device is integrated partially or entirely in a flexible fabric at a place where there are non-flexible components. All of these possible constructions are intended to be covered within the meaning of the invention.

According to an advantageous embodiment, the heat exchanger, during operation, keeps the temperature of the PCM below and/or within the melting range. In this way, the cooling effect of the PCMs is maintained, and also the temperature of the PCMs surrounding the body part is kept more or less constant, with the result that there are no undesired and abrupt temperature changes.

The device for temperature reduction, in particular also the heat exchanger, is particularly preferably integrated within the article, for example in the form of a double wall through which coolant flows, or in the form of a nonwoven with grooves along which coolant streams. Likewise, the PCMs are not necessarily provided across the whole surface, and instead it is possible to equip individual areas of the article with PCM “packs”, and in these cases the heat exchanger can advantageously also be limited to these areas of the article. As regards the integration of the PCM and the heat-exchanger function in the articles, it is not possible to give a comprehensive description, since the possibilities depend, firstly, on the article and, secondly, also on the wearer.

Provision is also made that the device for temperature reduction is also secured detachably on the article, such as a helmet, shoe or saddle, for example by a strap or velcro fastener. In this way, the device can be attached to the article or removed from the article depending on the time of year and on requirements.

A heat exchanger is a complete device comprising heat-absorbing and heat-emitting components. In the present case, this unit is composed of PCM element and of a space which bears on the PCM and through which the heat carrier flows. The flow can take place in an open system or as a closed circulation system. In the open system, the heat carrier medium is freely suctioned, conveyed past the PCM and then released to the environment. In a closed system, the heat is not given off directly to the environment, but indirectly via a further intermediate heat exchanger.

For example, heat exchangers that are operated with ambient air can be operated using the open system. On the other hand, heat exchangers with special coolants, for example including water, are preferably operated using the closed system.

The device can comprise in the form of a flexible sheet material, wherein a flexible matrix is provided wholly or partially with PCM or PCM-containing areas, wherein means are provided by which a medium of a heat exchanger is brought into heat-transferring contact with the PCMs of the flexible matrix, such that heat can be carried away from the PCMs and/or delivered to the PCMs.

The means by which the medium of the heat exchanger can come into contact with the PCMs, and hereinafter also simply called “means”, comprise flow structures such as channels, or elevations with different profiles such as grooves, channels, for example including meandering channels, knobs, pyramids, or others. The pattern that they form can be of any desired kind and can also serve as advertising. The flow structures, i.e. the means, naturally influence the speed with which the medium flows across the PCMs.

According to an advantageous embodiment of the invention, the flow structures at least partially form the surface structure of a flexible matrix.

According to a further embodiment, the means comprise not only the flow structures but also a device by which the medium of the heat exchanger is moved.

According to an advantageous embodiment, the PCM is present in the form of a paraffin, a salt hydrate and/or suitable modifications, such as PCM/polymer compounds, PCM/silicate compounds, PCM/graphite compounds, etc. In another embodiment, these PCMs or modifications thereof can be incorporated into corresponding carrier structures, e.g. capillary fiber suction structures.

According to an advantageous embodiment of the invention, the heat carrier medium is gaseous, for example ambient air. Provision can be made that the carrier of the flexible sheet material itself causes, for example by inherent movement (e.g. shoe, saddle), the movement of the medium through the flow structures for removing the heat of the PCMs. On the other hand, a small ventilator or some other device can be provided that causes the movement/flow of the medium.

According to an advantageous embodiment, the flow of the medium is adjusted such that the temperature of the PCMs is kept below or within the melting range of the PCMs.

According to one embodiment of the invention, the areas with PCMs are small islands in a flexible matrix that forms a woven or nonwoven cooling fabric.

Thus, according to one embodiment, small islands with PCM-containing packs are installed in a flexible sheet material and/or nonwoven cooling fabric worn on the body, wherein the properties of the flexible sheet material are maintained.

As the material for the flexible matrix or nonwoven cooling fabric, it is possible to use all types of known cushioning materials. In addition to the already mentioned silicone, other examples are polyurethane, thermoplastic polymers, copolymers, polyethylene and others.

The flow structure can be configured such that a medium flowing along the surface is conveyed across the islands and/or areas with PCMs, such that heat exchange from the PCM to the medium preferably takes place there.

For example, in addition to the solid socket, a below-knee prosthesis generally also comprises a flexible silicone stocking which is used to stabilize the soft tissue parts and which is pulled over the body part, bears directly on the skin and engages in the socket. For example, this is a silicone stocking which, according to one embodiment of the invention, is offset with areas, for example round islands, of PCM-containing material, wherein the properties of the flexible silicone stocking are substantially maintained. The socket can be provided with a small ventilator, through which air is blown along the inner face of the socket and then flows along the flow structures of the silicone stocking offset with PCM areas. The structures that guide the air stream can be provided both on the inner face of the socket and also on the outer face of the insert, that is to say of the silicone stocking, for example.

According to the invention, the socket can then also comprise a device for temperature reduction. For this purpose, it can be designed with a double wall, at least in some areas, such that PCM and heat exchanger system can be integrated practically in the socket. The means for moving the coolant, for example a ventilator or a small pump, can then be used together both for the nonwoven cooling fabric and also for the heat exchanger integrated in the solid part of the article, for example in the socket.

The means by which the medium of the heat exchanger comes into heat-conducting contact with the PCMs also comprise a device for generating a flow, for example a ventilator, pump or an opening in the shoe or saddle, which device pumps air through the shoe and the flow structures during movement. The flow structures have the effect that the medium flows past the PCMs. They can be provided on the flexible matrix with the PCMs or also separately therefrom, i.e. externally. Thus, these flow structures can also be provided on the inner face of the socket of a prosthesis, on the inner face of a shoe or of another rigid article that is in contact with the body of a living being. The means are configured such that the medium of a heat exchanger is brought by them into heat-transferring contact with the PCMs of the flexible sheet material.

Provision can be made that areas of the flexible matrix or of the article do not comprise a device for temperature reduction, i.e. are not provided with PCM-containing areas or islands, and/or that these areas of the article or of the flexible matrix are not provided with flow structures and, accordingly, the medium does not flow around them.

According to a further embodiment, the flexible sheet material comprises several layers, i.e. a stack, wherein, for example in miniaturized form, the islands with PCMs alternate with a flexible matrix. A wide variety of properties of the flexible sheet material can be obtained in this way. In particular, the side of the stack facing toward the body can have other surface properties than on the outwardly facing side of the stack. The material of the flexible matrix, and the PCMs too, can vary within one layer and within the stack.

For example, a layered structure can be obtained as follows:

Flexible matrix with PCM areas,

on top of the latter a layer with elevations that form flow channels for the medium of the heat exchanger,
on top of the latter a PCM layer, and then in turn a heat exchanger medium layer,
wherein the flexibility of the layers and of the overall configuration is maintained as far as possible despite the structuring and, according to the intended patterns of movement, can even support these.

For example, there are multi-layer silicone stockings that are used to stabilize soft-tissue parts on the body and that can serve here as flexible matrix. The PCMs, for example in the form of flat and small film cut-outs, are then incorporated into, cut into or applied to this flexible matrix in discrete areas, such that the mechanical properties such as flexibility and tear strength of the flexible matrix are substantially maintained. The incorporation of the PCMs into the flexible matrix depends on the respective materials, both of the matrix and also of the PCM, although every type of incorporation of the PCMs into a flexible matrix is intended to be covered within the meaning of the invention.

In addition to ambient air and other gaseous media as heat exchanger medium, it is also possible, for example, to provide a liquid heat exchanger medium or simply water.

Although the ambient air serves primarily as heat exchanger medium, the heat exchanger can be operated as an open and also as a closed system, wherein the medium coming into thermal contact with the PCMs is preferably, but not necessarily, regenerated continuously. “Regeneration” of the medium is to be understood primarily as meaning that the medium releases the heat taken up from the body via the PCM.

For example, the heat exchanger comprises a hose system, which is surrounded by PCM and through which water or ambient air is guided.

It is particularly preferable that the hose system itself, for example the material from which the hose is made, comprises the PCMs.

For example, the heat exchanger also comprises an annular gap of a double-walled article.

According to a further embodiment, the heat exchanger comprises micro-holes or micro-gaps through which ambient air flows or is pumped.

Here, examples of the “articles in direct thermal contact with the body of a living being” are helmets, prostheses, orthoses, shoes, saddles (of bicycles and on horses), car seats, gloves, all types of textiles, surfing suits, diving suits, spectacles and similar. Everything that generates built-up body heat is intended to be covered within the meaning of the invention.

Phase change material includes all types of commercially available PCMs that are capable of storing latent heat.

Storage of latent heat is understood as the storage of heat in a material that experiences a phase transition, mainly solid-liquid, i.e. a phase change material. In addition to the solid-liquid phase transition, it is also possible in principle to use solid-solid phase transitions. However, these generally show much lower storage densities.

When heat is stored in the storage material, the material begins to melt upon reaching the phase transition temperature and then, despite storing further heat, does not increase its temperature until the material has fully melted. Only then does an increase in the temperature occur again.

Since no appreciable temperature increase occurs for quite a long period of time despite delivery of heat, the heat stored during the phase transition is called “latent heat”. In the case of a solid-liquid phase transition, the latent heat is contained in the heat of fusion or heat of solidification of the storage material.

Storage of latent heat can take place in different temperature ranges. Depending on the particular use, a material having a suitable temperature of the phase change has to be chosen. Different classes of material are used according to the temperature range.

For the range of body heat, paraffins or salt hydrates, or modifications thereof, are generally proposed as PCMs within the meaning of the invention.

Thus, for example, PCMs can also be present in the form of compounds. Embodiments of these are paraffin/polymer compounds, salt hydrates, salt hydrate/graphite compounds, paraffin/silicate compounds, etc.

Paraffin/polymer compounds can also be present as a film, granulate, hose or other shaped bodies. In a further embodiment, these PCMs or modifications thereof can be incorporated into corresponding carrier structures, e.g. capillary fiber suction structures.

Other preferred variants for PCM and PCM compounds are film-packed PCMs.

Here, the preferred medium for the heat exchanger is ambient air and/or water, such that the device for temperature reduction functions solely by ambient air or water flowing through the conduit system surrounding the PCMs.

The medium can be subjected to regeneration or cooling, for example by being connected to a mobile cooling unit.

According to another embodiment, the medium is water, for example, which flows through the conduit system and is regenerated again by an associated cooling unit.

Another embodiment for additional cooling is composed of a material that can be saturated with liquid, e.g. a nonwoven that can be charged with water, for example, or other liquids that can evaporate. As a result of the evaporative cooling of the liquid by the stream of air forced past it, this nonwoven ensures a greatly reduced temperature of the cooling air.

A preferred embodiment of the evaporation nonwoven is outside the socket, e.g. on the suction side of the fan.

The invention is explained in more detail below on the basis of selected examples:

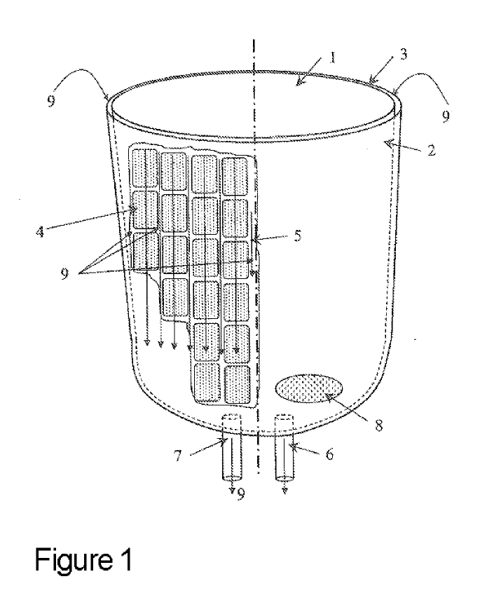

FIG. 1 shows the example of a leg or arm prosthesis or orthosis with a device for temperature reduction according to the present invention,

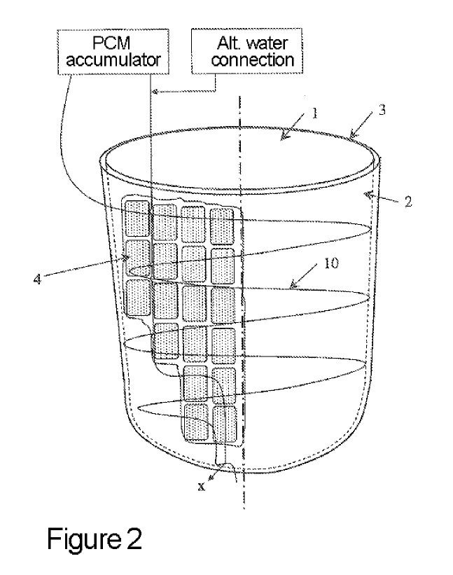

FIG. 2 shows a further prosthesis in which the device is integrated, and

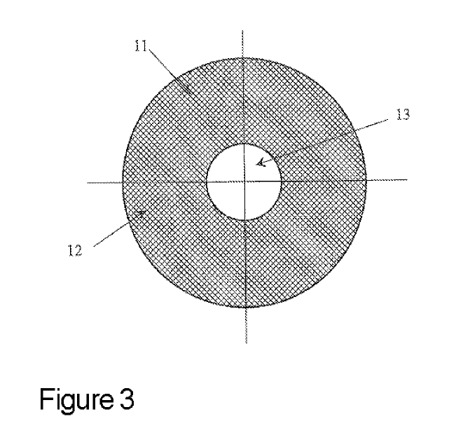

FIG. 3, finally, shows the cross section through a hose, wherein a compound is used as material of the hose.

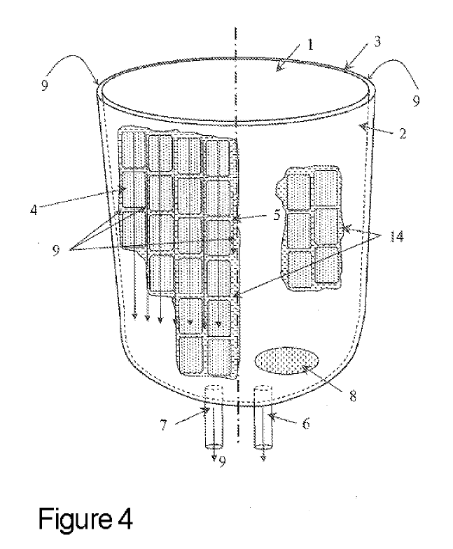

FIG. 4 shows the same view as FIG. 1, wherein heat of evaporation at least supports the regeneration of the PCMs.

FIG. 1 shows a prosthesis into which, in the case of an amputation, a so-called stump, i.e. the remaining part of the amputated limb, is inserted. These prosthesis are normally made of resin having a single wall.

According to the embodiment of the invention shown here, the single-wall resin construction is replaced by a double-wall construction, wherein the two walls 1 and 2, i.e. the inner wall 1 and the outer wall 2, define a hollow space that forms an annular gap 3. Here, as is shown, the annular gap 3 is open all the way round at the top. However, the annular gap can also be only partially open. As is shown here, PCM modules 4 are incorporated as discrete packs in the annular gap 3 and are arranged, for example, on a surface 5, for example a rubber surface. At the bottom of the prosthesis are, on the one hand, the suction valve 6 that maintains the underpressure in the prosthesis and, on the other hand, between the two walls 1 and 2, the suction valve 7 through which coolant that flows through the annular gap 3 around the PCM modules 4 is suctioned off. The arrows 9 indicate the path of the gaseous coolant through the annular gap 3 around the PCM modules 4. The valve 7 is connected, for example, to a pump, to a ventilator and/or to an external cooling unit (not shown) by which the air or the coolant is conveyed, suctioned, pumped and/or cooled through the annular gap 3, and as a result of which the PCM modules emit heat, or as a result of which moisture is taken up from the orthosis.

Accordingly, the heat exchanger is operated here as an open system since, for example, ambient air is simply sucked through the open annular gap 3 by the valve 7.

According to a preferred embodiment, a temperature sensor, by which the internal temperature can be established, is also located on the inner wall 1 of the prosthesis. The temperature sensor is connected to an external regulation and control unit that regulates the flow and/or the temperature of the coolant.

According to the embodiment of the invention shown in FIG. 1, micro-holes 8 are also provided in the outer wall 2 of the prosthesis and serve to convey further coolant, for example in the form of ambient air, through the annular gap 3. The micro-holes are shown here only by way of example, and they can of course be arranged in any desired manner, both in terms of the size of the arrangement and also in terms of the shape.

The PCM modules can be present as discrete packs or a charge of paraffin/polymer compound. However, they can also be present in any other desired form, and also as a simple charge in the annular gap closed at the bottom. Finally, the inner wall 2 can be composed wholly or partially of a compound comprising a PCM and a plastic in the form of a polymer, which also contains a flexible matrix for example.

According to the invention, the PCM modules 4 can likewise be distributed in any desired manner in the annular gap 3. For example, if less cooling is needed at the top and more is needed at the bottom, the density of the PCM modules can vary in any desired manner over the annular gap 3. Finally, the PCM modules can also be arranged to be movable and/or detachable, such that modules can be shifted or additional modules can be added or taken away.

FIG. 2 shows, like FIG. 1, the example of an orthosis or prosthesis. The double-wall construction with inner wall 1 and outer wall 2 can be seen again. However, in contrast to FIG. 1, the coolant here is not guided through an open annular gap 3, and instead there is a hose winding 10, which is placed between the PCM modules 4 and in which the coolant of the heat exchanger flows. The hose winding as heat exchanger is not necessarily a closed system and can, for example, be operated with air as coolant such that the ends of the cooling air that is blown through end in the interior of the orthosis/prosthesis. On the other hand, a liquid medium, for example water, can also be used as coolant, and the heat exchanger is then operated as a closed system with the hoses being returned to a water connection, a further heat exchanger or a water cooling system (external, not shown).

FIG. 3, finally, shows a cross section through a hose 11 which, according to the invention, can be used together with a double-wall system, in which PCM modules are present, or also without further PCM modules. The hose 11 is characterized by the fact that it forms a channel 13 for the coolant, and the hose 11 carries a compound with PCM 12, which compound, for example, contains a PCM in a polymer matrix.

A hose as shown in FIG. 3 can be produced in different thicknesses and can be integrated in all types of rigid and flexible articles. Many different requirements can thus be met, since the diameter of the hose wall, the internal diameter of the flow space and, finally, the compound into which the PCM is incorporated can be freely chosen.

FIG. 4 shows a further embodiment of the cold generation, in which the cooling air, conveyed by a fan for example, is guided according to the arrows 9 in the socket and through a nonwoven 14 saturated with water for example, and a greatly reduced temperature of the cooling air is ensured by the evaporative cooling of the liquid of the nonwoven by the stream of forced air that flows past it.

By way of example, the use in the soles of shoes is once again briefly outlined here. Instead of a complicated pumping or suctioning device, the walking movement can in this case simply activate the pumping of the coolant through the hose. The design as a PCM-containing hose can be used in many different ways; the hose can be incorporated wherever the external devices for moving the coolant replace physical movement of the body.

The invention discloses a device composed of PCM and of an actively cooling heat-exchanger system whose use in humans and animals reliably solves the previous problem of temperature build-up. The system can be configured in any strength, that is to say ranging from integration in flexible stockings to integration in orthosis sockets. This results in the extremely varied areas of application of the described technology, ranging from PCMs in the form of modules in coolant channels or in the form of PCM-containing compounds, from which hoses are produced in which coolant can flow, to PCMs with heat exchangers for removing the built-up heat of the body.

1. A device for temperature reduction, provided for articles or flexible sheet materials that are in direct thermal contact with the body of a living being, wherein a phase change material (PCM) and a heat exchanger are at least partially integrated in the article or the flexible sheet material, and the functionality of the article is maintained, wherein the PCMs are in heat-transferring contact with the heat exchanger.

2. The device as claimed in claim 1, wherein the heat exchanger carries a medium that keeps the temperature of the PCM below and/or within the melting range.

3. The device as claimed in either of claims 1 and 2, wherein the PCM is present in the form of PCM-containing modules and/or from a charge of PCM-containing particles.

4. The device as claimed in one of the preceding claims, wherein the PCM is preferably present from paraffin, salt hydrate and modifications thereof, e.g. as compounds.

5. The device as claimed in claim 4, wherein the compound is present in the form of a hose or PCM-filled pad.

6. The device as claimed in one of the preceding claims, wherein the PCM or the modifications thereof are incorporated into a carrier structure, e.g. fiber suction structure.

7. The device as claimed in one of the preceding claims, wherein the heat exchanger is present in the form of a hollow space through which coolant flows.

8. The device as claimed in one of the preceding claims, wherein the heat exchanger operation is open.

9. The device as claimed in one of the preceding claims, wherein ambient air is used as medium of the heat exchanger.

10. The device as claimed in one of the preceding claims, wherein the flexible sheet material is a multi-layer material.

11. The device as claimed in claim 10, wherein the sheet material comprises a silicone layer.

12. The device as claimed in either of claims 10 and 11, wherein the flow structures of the means by which the medium of the heat exchanger comes into heat-transferring contact with the PCMs are provided as a surface structuring of the silicone layer.

13. The device as claimed in either of claims 10 and 11, wherein the flow structures of the means by which the medium of the heat exchanger comes into heat-transferring contact with the PCMs are provided externally.

14. The use of PCMs in pure form and/or in the form of compounds with plastic polymers, coupled to a heat exchanger, in articles such as orthoses and/or prostheses, in items of clothing, in shoes, helmets, saddles or seats and/or as insoles or stockings.
